(19) United States
(12) Patent Application Publication
(10) Pub. No.: US 2009/0023700 A1
(43) Pub. Date: Jan. 22, 2009

(54) NEUROPROTECTIVE TREATMENTS

(75) Inventor: Theodoor Hagg, Louisville, KY (US)

Correspondence Address: VIKSNINS HARRIS & PADYS PLLP, P.O. BOX 111098, ST. PAUL, MN, 55111-1098, US

(73) Assignee: University of Louisville Research Foundation, Louisville, KY (US)

(21) Appl. No.: 12/176,152

(22) Filed: Jul. 18, 2008

Related U.S. Application Data
(60) Provisional application No. 60/951,092, filed on Jul. 20, 2007.

(51) Int. Cl. A61K 31/555; A61P 25/00

(52) U.S. Cl. 514/185

(57) ABSTRACT

The present invention provides a therapeutic method for treating traumatic neural injury or a degenerative disorder in a mammal by administering a neuroprotective compound, wherein the neuroprotective compound is a peroxovanadium compound, a peroxovandium derivative, or a compound with inhibitory activity of protein tyrosine phosphatases.

### RELATED APPLICATION(S)

This patent document claims the benefit of priority of U.S. application Ser. No. 60/951,092, filed Jul. 20, 2007, which application is herein incorporated by reference.

### GOVERNMENT FUNDING

This invention was made with government support under Grant Numbers RR15576 and NS045734 awarded by the National Institutes of Health. The government has certain rights in the invention.

### BACKGROUND OF THE INVENTION

Protection of the nervous system, such as the brain, sensory and motor systems and spinal cord in mammals during the acute and sub-acute phase of a injury to the nervous system, prior to or during surgery, or to treat a degenerative disease, would lead to great improvement in the quality of life, with the retention of touch, appropriate pain responses, and control of various bodily functions.

Currently, there is a need for neuroprotective treatments.

### SUMMARY OF THE INVENTION

Certain embodiments of the present invention provide compounds that are neuroprotective. Accordingly, certain embodiments of present invention provide therapeutic methods for treating a traumatic neural injury or a degenerative disorder in a mammal (e.g., a human, cat, dog, horse, donkey, mule, cow, sheep, goat, camel, etc.) comprising administering to a mammal in need of such therapy an effective amount of a neuroprotective compound, wherein the neuroprotective compound is a peroxovanadium compound, such as the peroxovandium, potassium bisperoxo(1,10-phenanthroline)oxovanadate (V) [bpV(phen)]), a bpV(phen) derivative, another peroxovanadium or a compound with inhibitory activity of protein tyrosine phosphatases including potassium bisperoxo(pyridine-2-carboxylato)oxovanadate(V) [bpV(pic)], pervanadate, periodinates and dephostatins. In certain embodiments, the neuroprotective compound is administered directly into or around the injured tissue, is administered through delivery into the cerebrospinal fluid, or is administered intravenously.

In certain embodiments, the traumatic neural injury is a spinal cord injury, a brain injury, a peripheral nerve injury, an eye injury affecting the optic nerve fibers, or a skin burn. In certain embodiments, the traumatic neural injury is caused by an ischemic or hemorrhagic stroke. In certain embodiments, the degenerative disorder is a neuron, axon, or myelin disorder, or an oligodendrocyte disorder, such as multiple sclerosis or peripheral neuropathy. In certain embodiments, the neuroprotective compound is administered as a pre-treatment prior to surgery.

Certain embodiments of the present invention provides a neuroprotective compound as described for the manufacture of a medicament useful for the treatment of a traumatic neural injury or a degenerative disorder in a mammal, wherein the neuroprotective compound is a peroxovanadium compound, a peroxovandium derivative, or a compound with inhibitory activity of protein tyrosine phosphatases.

### BRIEF DESCRIPTION OF THE FIGURES

FIGS. 1A-1D. Model to test sensory axon protection after spinal cord contusion. A, Top, The T9 contusive spinal cord injury (SCI) results in secondary degeneration of primary sensory axons that project to the gracile nucleus (GN). Hindlimb sensory axons are anterogradely traced by injecting cholera toxin subunit B (CTB) into both sciatic nerves. Reagents are infused though a catheter placed in the CSF through an L5/6 lumbar puncture. Sensory-evoked potentials (SEPs) are recorded from an epidural electrode. Experimental designs: Infusions (black horizontal bar) lasted 7 or 28 d, and CTB was injected 3 d before histological processing. In one experiment, sensorimotor function was tested by grid walking (grid). In another, sensory function was tested by SEP induced from the hindlimb and confirmed by a dorsal column transection (TXN). B, Normal primary sensory projections to the gracile nucleus can be visualized by CTB tracing. Scale bar, 200 μm. C, The projections are reduced 7 d after contusion. D, With increasing spinal cord displacement by the impactor, more innervation is lost. The 0.3 mm displacement enables detection of beneficial, detrimental, or neutral effects of test reagents.

FIGS. 2A-2F. Protein tyrosine phosphatase (PTP) inhibition by bpV(phen) treatment rescues dorsal column sensory axons and white matter. The concentration of bpV(phen), the histological measure, the time after the spinal cord contusion when the treatment was started (start), and the infusion site (site) are indicated below the graph. A, After a 7 d infusion of 30 μM bpV(phen) at the T9 contusion site started immediately after the injury, more sensory axons remained intact than with PBS (0), as measured by the CTB-labeled terminal fiber area in the gracile nucleus. p<the values indicated above the columns. Values are expressed as a percentage of seven sham-operated rats. B, bpV(phen) also protected the dorsal column white matter (WM) at the injury epicenter. A dose-response study using 7 d infusions at L5/6 started immediately after the injury showed that 100 μM bpV(phen) was most effective in protecting gracile nucleus innervation (C, CTB) and T9 dorsal column white matter (D, WM). When started 4 h after the contusion, 7 d L5/6 bpV(phen) infusions also rescued gracile nucleus innervation (E) and T9 dorsal column white matter (F).

FIGS. 3A-B. PTP inhibition provides lasting functional benefits after spinal cord contusion. A, PBS or PBS with 100 μM bpV(phen) was infused from L5/6 for 28 d, starting 4 h after a contusion at T9. Before the spinal cord contusion, the groups had similar baseline values in the grid-walk test. After the T9 spinal cord contusion, the bpV(phen)-infused group had fewer hindlimb footfalls and reached normal levels and that the major effect is during the first week. The columns on the right show that peroxovandadium-treated rats completed the task quicker. p<the numbers over the data points. B, bpV(phen)-treated rats responded to the same Semmes-Weinstein filament sizes applied to the trunk as the PBS rats.

FIGS. 4A-4G. PTP inhibition provides lasting protection of dorsal column sensory axons and white matter after a spinal cord contusion. The rats infused for 28 d starting 4 h after contusion (FIG. 3) were analyzed 2 weeks later. Compared with sham-operated rats (A), injured ones infused with PBS (B) showed a reduction in CTB-traced innervation from the hindlimb to the gracile nucleus. C, bpV(phen)-infused rats had an apparently normal innervation (compare also with FIG. 1 B). Scale bar, 500 μm. The number of myelinated axons in the fasciculus gracilis (g in the inset) at C3 was reduced in PBS-infused rats (E) compared with sham rats (D). Arrows, Examples of myelin debris. F, The bpV(phen)-treated rats had an apparently normal number of axons. c in the insets, Fasciculus cuneatus. Scale bar, 20 μm. Injury-induced loss of white matter at the injury epicenter (H, PBS-infused rat) was reduced after infusion of bpV(phen) (I) to sham levels (G). Note the absence of the injury-induced central cavitation (*) in the bpV(phen)-treated rat. Scale bar, 500 μm.

FIGS. 5A-5B. PTP inhibition provides lasting protection: quantification. A, Six weeks after the contusion and 2 weeks after the termination of the infusion, the CTB-traced innervation of the gracile nucleus (GN CTB) was greater with infusion of 100 μM bpV(phen) (100 bpV) than with PBS treatment and comparable with the sham values. The number of axons in the fasciculus gracilis at C3 was completely protected by bpV(phen). The dorsal column white matter at the injury epicenter (epi) was protected to 80% of sham. B, The number of axons at C3 predicted the performance in the last grid-walk test 6 d before. The regression analysis was performed on data pooled from both treatment groups. The p value indicates the significance of the correlation between the axon counts and the grid performance. Open squares, PBS; filled circles, bpV(phen).

FIGS. 6A-6C. PTP inhibition rescues dorsal column sensory-evoked potentials after spinal cord contusion. A, An example of evoked potentials (arrows) recorded from the gracile nucleus of a rat. The SEP is evident before a spinal cord contusion, much reduced 14 d after injury, and absent after selective transection of the dorsal column (DC Txn) to confirm the specific role of the latter. Traces represent the average of 20 measurements. The SEP is expected between 11 and 15 ms after the stimulus artifact (*). B, SEP amplitudes of individual rats in the PBS-infused group shows the loss of SEPs after the contusion in all but one rat, as well as the disappearance of the SEP after dorsal column transection. C, All rats in the bpV(phen)-infused group had a measurable SEP after the contusion, and all SEPs disappeared after the dorsal column transection. The sham values (open triangles, solid line) are shown for comparison.

FIGS. 7A-7D. PTP inhibition reduces epicenter inflammation and blood vessel loss after spinal cord contusion. A, B, Iba1 immunostaining of transverse sections through the dorsal part of the spinal cord including the dorsal column (DC) and dorsal horns (DH) show microglial/macrophage activation 7 d after a spinal cord injury in PBS-infused rats (A) and reduced activation with bpV-(phen) infusions (B). Infusions were started 4 h after the injury. The PBS-infused rat shows infiltrates of activated macrophages, whereas the bpV(phen)-infused rat shows predominantly activated microglial cells, judged by their processes and less rounded appearance. In the same sections, immunostaining for the endothelial marker RECA1 shows loss of blood vessels in PBS-infused rats (C) but an almost normal appearance in bpV(phen)-treated rats (D). Asterisk indicates central canal.

FIGS. 8A-8F. PTP inhibition may rescue axons by rescuing epicenter blood vessels. A, Quantification of the area of Iba1 as a measure of microglia/macrophage activation 7 d after spinal cord injury shows a reduction in the bpV(phen)-treated group (bpV) only at 1 mm rostral to the injury epicenter. Only the rats in which the infusion was started 4 h after the injury were included. B, EC staining measuring spared epicenter white matter shows a clear correlation between white matter loss and Iba1. C, The extent of loss of CTB-labeled projections only correlated with Iba1 at 1 mm rostral to the epicenter. The epicenter and caudal levels are not shown. D, Analyses of immunostaining for the endothelial cell marker RECA1 in the dorsal column of the same sections shows that bpV(phen) rescues blood vessels at the epicenter. E, To increase the power of regression analyses, epicenter RECA1 was measured also in the rats from the dose-response (DR) study in which PBS or bpV(phen) infusions were started immediately after the injury and lasted for 7 d. BpV(phen) increases the area when given at 30 or 100 μM starting immediately after the injury. The 4 h delayed treatment groups in D are shown again for comparison. F, Regression analysis for the combined values of all experimental groups from E shows a correlation between the area of dorsal column blood vessels (RECA1) and the extent of dorsal column axonal projections to the gracile nucleus (CTB).

FIGS. 9A-9D. PTP inhibition rescues serum-deprived endothelial cells in vitro. A, Purified human aorta endothelial cells cultured for 6 h under serum-deprived conditions (0 or 5% serum instead of 10-20) undergo apoptosis without addition of bpV(phen) (0). With 3 μM bpV(phen), fewer cells have apoptotic nuclei. B, After 18 h of culturing with various degrees of serum deprivation (0, 1.25, 2.5, and 5% serum), more endothelial cells undergo apoptosis without bpV(phen), but addition of 3 μM bpV-(phen) during that time rescues the cells. The 1 μM bpV(phen) treatment only reduces endothelial apoptosis in the 2.5 and 5% serum groups. These data suggest that bpV(phen) can directly rescue endothelial cells in vitro, as it does in vivo after spinal cord injury.

### DETAILED DESCRIPTION

Nervous system injury, e.g., spinal cord injury, causes progressive secondary tissue degeneration, leaving many injured people with neurological disabilities. There are no satisfactory neuroprotective treatments. Protein tyrosine phosphatases inactivate neurotrophic factor receptors and downstream intracellular signaling molecules. Whether the peroxovanadium compound bpV(phen) [potassium bisperoxo(1,10-phenanthroline)oxovanadate (V)], a stable, potent and selective protein tyrosine phosphatase inhibitor, would be neuroprotective after a thoracic spinal cord contusion in adult rats was evaluated. Intrathecal bpV(phen) infusions through a lumbar puncture rescued dorsal column sensory axons innervating the nucleus gracilis and white matter at the injury epicenter. At the most effective dose, essentially all of these axons and most of the white matter at the epicenter were spared (vs ˜60% with control infusions). bpV(phen) treatments that started 4 h after contusions were fully effective. This treatment greatly improved and normalized sensorimotor function in a grid-walking test and provided complete axonal protection over 6 weeks. The treatment rescued sensory-evoked potentials that disappeared after dorsal column transection. bpV(phen) appeared to affect early degenerative mechanisms because the main effects were seen at 7 d and lasted beyond the treatment period. The neuroprotection appeared to be mediated by rescue of blood vessels. bpV(phen) reduced apoptosis of cultured endothelial cells. These results show that a small molecule, used in a clinically relevant manner, reduces loss of long-projecting axons, myelin, blood vessels, and function in a model relevant to the most common type of spinal cord injury in humans. They reveal a novel mechanism of spinal cord degeneration involving protein tyrosine phosphatases that can be targeted with therapeutic drugs.

The bpV(phen) treatment also greatly reduced degeneration of myelin at the injury site. Such myelin degeneration is caused by death of oligodendrocytes, which make the myelin, and by direct attack on myelin. An important contributor to myelin loss after injury is the inflammation during the sub-acute phase of the injury. Multiple sclerosis is characterized by myelin degeneration caused by very similar inflammatory processes. Therefore, a neuroprotective peroxovanadium treatment would also reduce neurological deficits if given to a person with multiple sclerosis.

Accordingly, certain embodiments of the invention provide therapeutic methods for treating traumatic neural injury in a mammal, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

Certain embodiments of the invention provide therapeutic methods for treating a degenerative disorder in a mammal, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

Certain embodiments of the invention provide therapeutic methods for pre-treating a mammal prior to surgery to prevent injury to nerves, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

Certain embodiments of the invention provide therapeutic methods for preventing damage to endothelial cells, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

Certain embodiments of the invention provide therapeutic methods for preventing damage to blood vessels, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

In certain embodiments, the neuroprotective compound is potassium bisperoxo(1,10-phenanthroline)oxovanadate (V) [bpV(phen)].

In certain embodiments, the neuroprotective compound is potassium bisperoxo(pyridine-2-carboxylato)oxovanadate(V) [bpV(pic)], pervanadate, periodinate or dephostatin.

In certain embodiments, the neuroprotective compound is administered directly into or around the injured tissue, is administered through delivery into the cerebrospinal fluid, is administered onto or directly into the eye or is administered intravenously.

In certain embodiments, the neuroprotective compound is administered at a concentration of about 100 μM.

In certain embodiments, the neuroprotective compound is administered at a concentration of less than 100 mM.

In certain embodiments, the neuroprotective compound is administered at a concentration of about 30 to 300 μM.

In certain embodiments, the neuroprotective compound is administered at a concentration of less than about 100 μM.

In certain embodiments, the neuroprotective compound is administered within about 0-48 hours of injury.

In certain embodiments, the neuroprotective compound is administered within about 2-24 hours of injury.

In certain embodiments, the neuroprotective compound is administered within about 3-12 hours of injury.

In certain embodiments, the neuroprotective compound is administered within about 3-5 hours of injury.

In certain embodiments, the mammal is a human, cat, dog, horse, donkey, mule, cow, sheep, goat, or camel.

In certain embodiments, the neuroprotective compound is administered for a period of about one to four weeks.

In certain embodiments, the traumatic neural injury is a spinal cord injury, brain injury, a peripheral nerve injury, an eye injury affecting the optic nerve fibers, or a skin burn.

In certain embodiments, the injury, e.g., the traumatic neural injury, is a traumatic spinal cord injury.

In certain embodiments, the injury, e.g., the traumatic neural injury, is caused by an ischemic or hemorrhagic stroke.

In certain embodiments, the degenerative disorder is a neuron, axon, or myelin disorder.

In certain embodiments, the degenerative disorder is an oligodendrocyte disorder.

In certain embodiments, the degenerative disorder is multiple sclerosis or peripheral neuropathy.

Certain embodiments of the invention provide the use of a neuroprotective compound as described for the manufacture of a medicament useful for the treatment of a traumatic neural injury, or a degenerative disorder in a mammal, or for pre-treating a mammal prior to surgery to prevent injury to nerves, or to prevent damage to endothelial cells, wherein the neuroprotective compound is a peroxovanadium compound, a peroxovandium derivative, or a compound with inhibitory activity of protein tyrosine phosphatases.

Certain embodiments of the invention provide the use of a peroxovanadium compound, a peroxovanadium derivative, or a compound with inhibitory activity of protein tyrosine phosphatases for treating a traumatic neural injury.

Certain embodiments of the invention provide the use of a peroxovanadium compound, a peroxovanadium derivative, or a compound with inhibitory activity of protein tyrosine phosphatases for treating a degenerative disorder.

Certain embodiments of the invention provide the use of a peroxovanadium compound, a peroxovanadium derivative, or a compound with inhibitory activity of protein tyrosine phosphatases for pre-treating a mammal prior to surgery to prevent injury to nerves.

Certain embodiments of the invention provide the use of a peroxovanadium compound, a peroxovanadium derivative, or a compound with inhibitory activity of protein tyrosine phosphatases for preventing damage to endothelial cells.

Certain embodiments of the invention provide the use of a peroxovanadium compound, a peroxovanadium derivative, or a compound with inhibitory activity of protein tyrosine phosphatases for preventing damage to blood vessels.

Therapeutic Compositions

The neuroprotective compound used in the methods described herein can be a peroxovanadium compound (Posner et al., J. Biol. Chem. 269:4596-4606 (1994)), such as the peroxovandium, potassium bisperoxo(1,10-phenanthroline)oxovanadate (V) [bpV(phen)]), or a bpV(phen) derivative, or another peroxovanadium or a compound with inhibitory activity of protein tyrosine phosphatases, which includes potassium bisperoxo(pyridine-2-carboxylato)oxovanadate(V) [bpV(pic)], pervanadate, periodinates (Leung et al., Bioorg & Med. Chem. 9:353-356 (1999)), and dephostatins. The neuroprotective compounds can be formulated as pharmaceutical compositions and administered to a mammalian host, such as a human patient, in a variety of forms adapted to the chosen route of administration. The neuroprotective compound could also be administered to other types of mammals in need thereof, such as dogs, cats, horses, donkeys, mules, cows, sheep, goat, and camels. The neuroprotective compound may be administered, e.g., intrathecally, intraocularly, intravenously or intraperitoneally by infusion or injection. Solutions of the active compound or its salts can be prepared in phosphate buffered saline or saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. Some of the compounds used in the present invention are not stable after dissolving them, so a dry formulation would need to be activated by bringing it into solution before administration.

The pharmaceutical dosage forms suitable for injection or infusion include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the neuroprotective compound can be determined by comparing their in vitro activity, and in vivo activity in animal models. Methods for the extrapolation of effective dosages in mice, and other animals, to humans are known to the art; for example, see U.S. Pat. No. 4,938,949.

The desired dose may conveniently be presented in a continuous or single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention also provides a kit comprising a neuroprotective compound, or a pharmaceutically acceptable salt thereof, at least one other therapeutic agent, packaging material, and instructions for administering the compound of formula I or the pharmaceutically acceptable salt thereof and the other therapeutic agent or agents to an animal to treat traumatic neural injury or a degenerative disorder.

Concentrations and Duration of Treatment

The concentration of the neuroprotective compound will vary depending on the condition to be treated and/or the mode of administration.

In certain embodiments, the compound is administered for a period of less than six weeks. In certain embodiments, the compound is administered for a period of about one to four weeks. In other embodiments, such as to treat a degenerative disease such as multiple sclerosis, the compound will be administered for an extended period of time, such as for several years, or for the life of the patient.

Traumatic Spinal Cord Injury or Traumatic Brain Injury

For example, if the neuroprotective compound is administered to treat a traumatic spinal cord injury or traumatic brain injury, then the neuroprotective compound can be administered at a dosage of less than about 10 mM, such as at a range of 1 nM to 10 mM or any integer value in between. For example the dosage may be at a range of about 30 to 300 μM, or of about 100 μM to 300 μM. In one embodiment, the neuroprotective compound is administered at a concentration of about 30 μM or about 100 μM. In such situations, the neuroprotective compound can be administered intrathecally or intravenously. In certain embodiments, the neuroprotective compound is administered within about 0-48 hours of injury. In certain embodiments, the neuroprotective compound is administered within about 2-24 hours of injury. In certain embodiments, the neuroprotective compound is administered within about 3-12 hours of injury. In certain embodiments, the neuroprotective compound is administered within about 3-5 hours of injury.

As used herein, the term “traumatic spinal cord injury” or “traumatic brain injury” is defined as encompassing a non-severing injury to the spinal cord or brain tissue. Examples of such injuries include a contusive injury, bruise, severe inflammation or other type of injury that does not cut the tissue.

Multiple Sclerosis

The neuroprotective compound may be administered to treat multiple sclerosis. To treat multiple sclerosis, the neuroprotective compound can be administered at a dosage of less than about 10 mM, such as at a range of 1 nM to 10 mM or any integer value in between. For example the dosage may be at a range of about of about 30 to 300 μM, or of about 100 μM to 300 μM. In such situations, the neuroprotective compound can be administered intrathecally or intravenously. When treating multiple sclerosis, the neuroprotective compound may be administered for an extended period of time, such as for a period of days, weeks or years, or for the lifetime of the patient. The neuroprotective compound may be administered as a series of injections.

Peripheral Nerve Injury

The neuroprotective compound may be administered to treat a peripheral nerve injury. If the neuroprotective compound is administered to treat a peripheral nerve injury, then the neuroprotective compound can be administered at a dosage of less than about 10 mM, such as at a range of 1 nM to 10 mM or any integer value in between. For example the dosage may be at a range of about of about 30 to 300 μM, or of about 100 μM to 300 μM. In such situations, the neuroprotective compound can be administered intrathecally, intravenously, or injected directly into the site of injury. In certain embodiments, the neuroprotective compound is administered within about 0-48 hours of injury. In certain embodiments, the neuroprotective compound is administered within about 2-24 hours of injury. In certain embodiments, the neuroprotective compound is administered within about 3-12 hours of injury. In certain embodiments, the neuroprotective compound is administered within about 3-5 hours of injury.

Eye Injury Affecting the Optic Nerve Fibers

The neuroprotective compound may be administered to treat an eye injury affecting the optic nerve fibers. If the neuroprotective compound is administered to treat an eye injury, then the neuroprotective compound can be administered at a dosage of less than about 10 mM, such as at a range of 1 nM to 10 mM or any integer value in between. For example the dosage may be at a range of about 30 to 300 μM, or of about 100 μM to 300 μM. In such situations, the neuroprotective compound can be administered intrathecally, intravenously, or injected directly into the site of injury. In certain embodiments, the neuroprotective compound is administered within about 0-48 hours of injury. In certain embodiments, the neuroprotective compound is administered within about 2-24 hours of injury. In certain embodiments, the neuroprotective compound is administered within about 3-12 hours of injury. In certain embodiments, the neuroprotective compound is administered within about 3-5 hours of injury.

Ischemic or Hemorrhagic Stroke

The neuroprotective compound may be administered to treat ischemic or hemorrhagic stroke. If the neuroprotective compound is administered to treat ischemic or hemorrhagic stroke, then the neuroprotective compound can be administered at a dosage of less than about 10 mM, such as at a range of 1 nM to 10 mM or any integer value in between. For example the dosage may be at a range of about 30 to 300 μM, or of about 100 μM to 300 μM. In such situations, the neuroprotective compound can be administered intrathecally, intravenously, or injected directly into the site of injury. In certain embodiments, the neuroprotective compound is administered within about 0-48 hours of injury. In certain embodiments, the neuroprotective compound is administered within about 2-24 hours of injury. In certain embodiments, the neuroprotective compound is administered within about 3-12 hours of injury. In certain embodiments, the neuroprotective compound is administered within about 3-5 hours of injury.

Skin Burn Injury

The neuroprotective compound may be administered to treat a skin burn. The neuroprotective compound may be administered topically, intrathecally, intravenously, or injected directly into the site of injury. The concentration of the neuroprotective compound may vary depending on the mode of administration. If the neuroprotective compound is administered topically, then the neuroprotective compound can be administered at a dosage of less than about 100 mM, such as at a range of 1 nM to 100 mM or any integer value in between. For example the dosage may be at a range of about 30 μM to 10 mM, or of about 100 μM to 1 mM. If the neuroprotective compound is administered intrathecally, intravenously, or injected directly into the site of injury, then the dosage can be at a concentration of less than about 1 mM, such as at a range of about of about 30 to 300 μM, or of about 100 μM to 300 μM. In certain embodiments, the neuroprotective compound is administered within about 0-48 hours of injury. In certain embodiments, the neuroprotective compound is administered within about 2-24 hours of injury. In certain embodiments, the neuroprotective compound is administered within about 3-12 hours of injury. In certain embodiments, the neuroprotective compound is administered within about 3-5 hours of injury.

Pre-Treatment Prior to Surgery

The neuroprotective compound may be administered as a pretreatment prior to surgery. The neuroprotective compound may be administered topically, intrathecally, intravenously, or injected directly into the site of surgery. The concentration of the neuroprotective compound may vary depending on the mode of administration. If the neuroprotective compound is administered topically, then the neuroprotective compound can be administered at a dosage of less than about 10 mM, such as at a range of 1 nM to 10 mM or any integer value in between. For example the dosage may be at a range of about 30 μM to 1 mM, or of about 100 μM to 300 μM. If the neuroprotective compound is administered intrathecally, intravenously, or injected directly into the site of injury, then the dosage can be at a concentration of less than about 1 mM, such as at a range of 1 nM to 1 mM or any integer value in between. For example the dosage may be at a range of about 30 to 300 μM, or of about 100 μM to 300 μM. In certain embodiments, the neuroprotective compound is administered within about 0-48 hours of surgery. In certain embodiments, the neuroprotective compound is administered within about 2-24 hours of surgery. In certain embodiments, the neuroprotective compound is administered within about 3-12 hours of surgery. In certain embodiments, the neuroprotective compound is administered within about 3-5 hours of surgery.

The invention will now be illustrated by the following non-limiting Example.

### EXAMPLE 1

Spinal cord injury causes immediate mechanical tissue damage, followed by secondary degeneration over a period of weeks, leading to loss of axons, myelin, blood vessels, and tissue at the injury site. Many long-projecting axons are not severed by the primary injury, particularly after contusions, and protecting them and their myelin should reduce the devastating loss of function. Contusions comprise ˜25-40% of human injuries. Few studies have convincingly shown therapeutic protection of axons of passage after a contusion injury.

Peroxovanadiums are a synthetic small molecule PTP inhibitor and enhances tyrosine phosphorylation and signaling in several systems (Posner et al (1994) J Biol Chem 269:4596-4604; Sekar et al. (1996) Crit Rev Biochem Mol Biol 31:339-359; and Ruff et al. (1997) J Biol Chem 272:1263-1267).

As described herein, whether PTP inhibition by bpV(phen) would reduce degeneration of primary afferent sensory axons that project through the dorsal column to the nucleus gracilis in the medulla and their white matter tracts after a contusive spinal cord injury was tested in adult rats. Lasting improvement in function as measured by behavioral and electrophysiological tests was determined. Whether the bpV(phen) treatment could be given in a clinically relevant manner, e.g., started 4 h after the primary injury, delivered via a lumbar puncture, given for a limited time, and lacking negative side effects such as pain was evaluated. The potential role of inflammation on endothelial cell survival was also investigated.

### Results

Dorsal Column Sensory Axon Injury Parameters

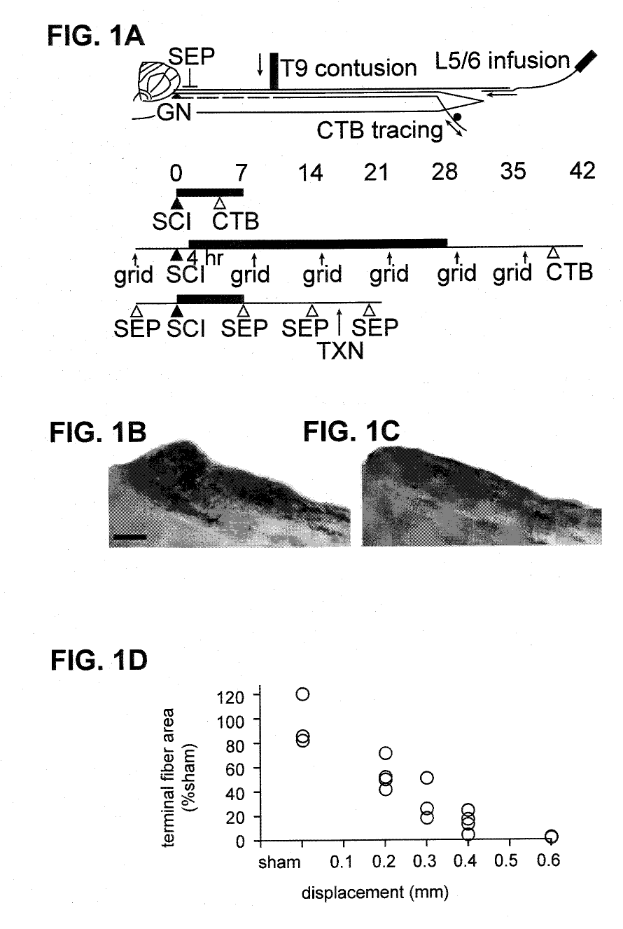

To enable efficient testing of whether drugs are neuroprotective, neutral, or detrimental, the injury severity that causes loss of two-thirds of the primary sensory dorsal column axons 7 d after injury was first determined (Baker et al. (2007) Exp Neurol 205:82-91). Rats received a contusion at spinal level T9 with the new Louisville Impactor System Apparatus using displacements of 0 (sham laminectomy), 0.2, 0.3, 0.4, or 0.6 mm (n=4 each) (FIG. 1A). Four days later, the sensory fibers and terminals were anterogradely labeled by injecting CTB into both sciatic nerves. Seven days after the injury, the sensory innervation of the gracile nucleus was reduced (FIG. 1C), with progressively fewer CTB-labeled fibers seen with increased injury displacement (FIG. 1D). Only 31+/−7% (percentage of sham +/−SEM) remained after a 0.3 mm injury, which was used in most of the experiments. At the 0.6 mm displacement, virtually none of the projections remained present in the gracile nucleus. The variability within each group was similar to that of the sham group, suggesting that the injury severity is consistent and contributes little to the variability of the CTB-labeled projections to the gracile nucleus.

Lumbar Intrathecal bpV(Phen) Infusions Protect Dorsal Column Sensory Axons and White Matter

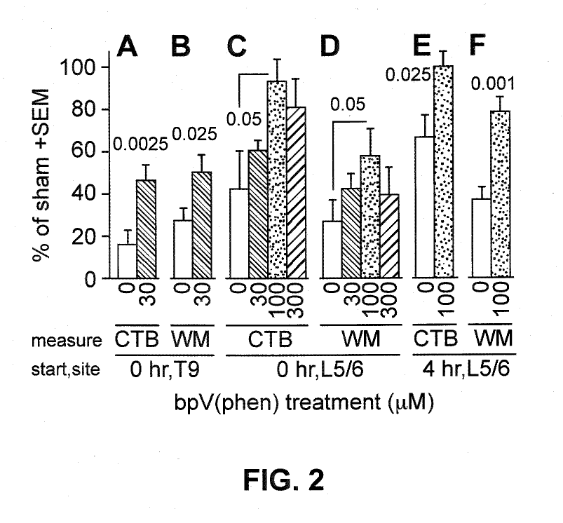

To test the neuroprotective effects of PTP inhibition, rats with a 0.3 mm T9 contusion received an intrathecal (subarachnoid) infusion for 7 d at the T9 injury site with PBS (n=9 from 2 separate experiments) or PBS with 30 μM bpV(phen) (n=9 from 2 separate experiments). The infusion was started immediately after the injury. With bpV(phen), 47+/−6% (of sham) of the CTB-labeled terminal fiber area was seen in the gracile nucleus compared with 17+/−7% with PBS (p<0.0025) (FIG. 2A). The CTB value of the PBS-infused group was not significantly different from that of the non-infused injured 0.3 mm displacement group. However, six of nine rats had very low values, possibly because of damage to the dorsal column axons by the dorsally positioned catheter from T11 to T9. The area of spared white matter in the dorsal column in transverse sections through the injury epicenter was also greater in the bpV(phen) (50+/−8% of sham; p<0.025) than in the PBS group (28+/−6%) (FIG. 2B). This demonstrates that local PTP inhibition by bpV(phen) infusion can reduce loss of sensory axons as well as their white matter by 7 d after injury.

To enhance the clinical relevance, the infusion catheter was placed into the CSF space at vertebral level L5/6, where there is no spinal cord. This mimics administration through a lumbar puncture in humans. To determine the lowest concentration leading to the maximum protection, rats with 0.3 mm T9 contusions were infused immediately after the injury with PBS (n=4) or PBS containing 30 μM (n=5), 100 μM (n=6), or 300 μM (n=5) bpV(phen) for 7 d. The CTB-traced fiber area in the gracile nucleus was 43+/−17, 60+/−5, 93+/−11 (p<0.05 vs PBS), and 80+/−14%, respectively (FIG. 2C). The value of the 100 μM group was not significantly different from that of the sham-operated rats, indicating the ability of bpV(phen) to provide complete protection. Of note is that the PBS values were higher in the rats infused at L5/6 (FIG. 2C) than at T9 (FIG. 2A), again suggesting that the catheter can be detrimental to sensory axons after being inserted into the subarachnoid space at T11 and its tip positioned at T9. The area of spared dorsal column white matter at the injury epicenter was also greatest in the 100 μM group (58+/−12%; p<0.05) (FIG. 2D). Total white matter at the epicenter was also protected in the 100 μM group (90+/−12 vs 68+/−6% with PBS; p<0.05). Based on these results, all following experiments used 100 μM bpV(phen). The epicenter white matter values in the lumbar-infused PBS group were similar to those seen in the T9-infused PBS group. This is consistent with the fact that the catheter tip was positioned just caudal to the epicenter and thus did not impinge on or cause damage to the epicenter itself.

bpV(phen) Treatment Retains Efficacy when Started 4 h after Spinal Cord Injury

Whether treatment could be delayed for 4 h, a time period within which most diagnoses of human spinal cord injury in developed countries can be made and within which time lumbar infusions could start in most trauma centers, was tested. Rats received a 0.3 mm T9 contusion, and a lumbar infusion with PBS (n=6) or PBS containing 100 μM bpV(phen) (n=7) was started 4 h later. Seven days later, the bpV(phen) group had a normal extent of gracile nucleus innervation (100+/−7%), which was greater than seen with delayed PBS infusions (66+/−11%; p<0.025) (FIG. 2E) and not significantly different from the sham value. The average of the delayed PBS group was significantly greater than without infusions in the first experiment (p<0.05), suggesting that the infusion per se has neuroprotective effects. The 4 h delayed bpV-(phen) treatment also increased spared dorsal column white matter at the epicenter (78+/−7 vs 37+/−6% PBS; p<0.001) (FIG. 2F). The value of the bpV(phen) group was not significantly different from that of the sham group (p<0.53). Total white matter was also protected by bpV(phen) (103+/−4 vs 70+/−5% with PBS; p<0.00025).

Delayed bpV(phen) Treatment Provides Lasting Improvement in Sensorimotor Function

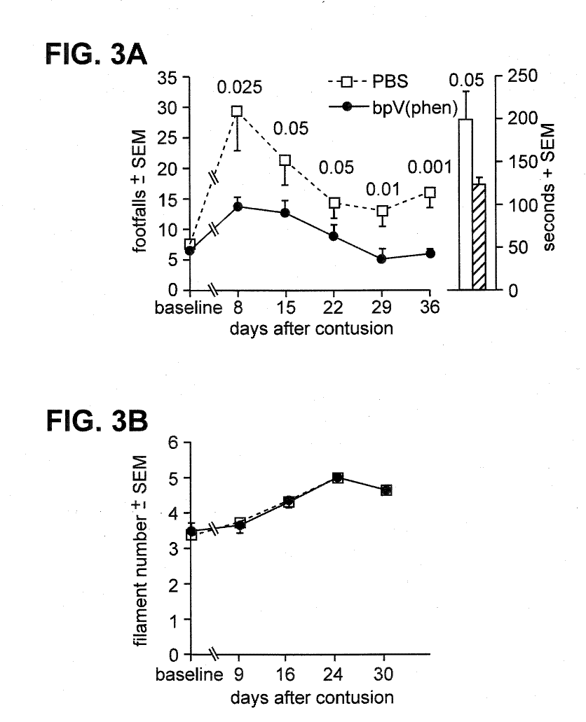

Whether the 4 h delayed treatment would have lasting neuroprotective effects resulting in functional benefits was tested. Rats received a 0.3 mm contusion at T9 and 4 h later were implanted with a lumbar catheter to start infusions with PBS (n=9) or PBS containing 100 μM bpV(phen) (n=9). The treatment was maintained for 28 d by replacing the Alzet pumps every week with ones containing fresh reagents (FIG. 1A). Weekly hindlimb grid-walking performance was used to test sensorimotor function, which is in part dependent on the primary afferents and second-order propriospinal axons of the fasciculus gracilis in the dorsal columns. Weekly tests showed that the bpV(phen) group performed better than the PBS group at all postinjury times (p<0.0000025 per ANOVA) (FIG. 3A). The greatest difference was seen at 8 d, during the first postinjury test, when the number of footfalls (sum of both hindlimbs) in the bpV(phen) group (14+/−2) was half of that in the PBS group (29+/−6; p<0.025) and only twice that seen before the contusion (7+/−1). Before the contusion, when the rats were still naive (baseline values), the number of footfalls was 3-14 and was not different between the groups. At day 8, seven of nine bpV(phen)-treated rats were within that normal range compared with only one of nine in the PBS group. Both groups reached plateau values by the 29 d test, with the bpV(phen) group reaching a normal average (6+/−1) and the PBS group being significantly higher (p<0.01; 13+/−2). The average of either group did not change after termination of the infusion, i.e., the 29 and 36 d values for each rat were not significantly different (paired t test). The bpV(phen)-treated rats performed the required 90 s of grid walking quicker than the PBS-treated rats, with a total time of 151 vs 219 s at 8 d (p<0.005) and 125 vs 200 s at 36 d (p<0.05) (FIG. 3A). Naive rats take ˜150 s to complete the task. Thus, the bpV(phen) treatment rescued both the agility and speed at which the grid-walk test was performed, bringing them back to normal performance levels.

Whether bpV(phen) might have detrimental side effects, such as inducing pain at the level of the injury, was tested. Severe spinal cord injury can induce hypersensitivity and mechanical allodynia (a painful response to a nonpainful tactile skin stimulus) at the level of injury. As expected, the moderate 0.3 mm injury did not induce allodynia. In fact, increasingly stiffer filaments were needed to evoke a response over time after the injury, suggestive of habituation (FIG. 3B). The values of the bpV(phen)-infused group were not different from those of the PBS group during the entire test period. Thus, chronic bpV(phen) infusions do not induce pain, an important feature of experimental treatments that would be considered for preclinical development. Differences between any of the bpV(phen)- and PBS-treated groups in the overall health status, general behavior, and grooming of the rats were not observed.

bpV(phen) Provides Lasting Protection of Dorsal Column Sensory Axons and White Matter

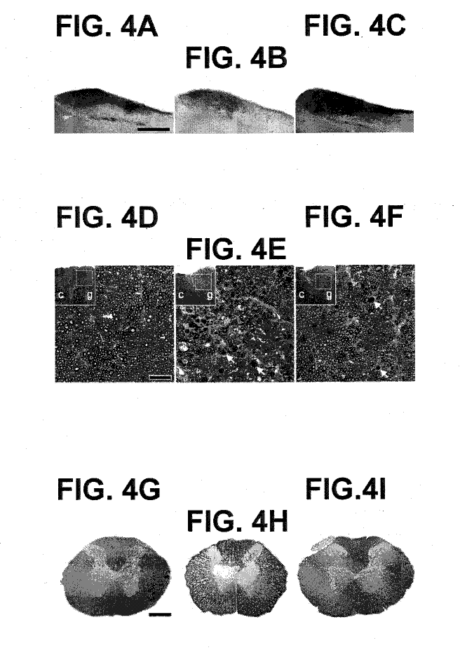

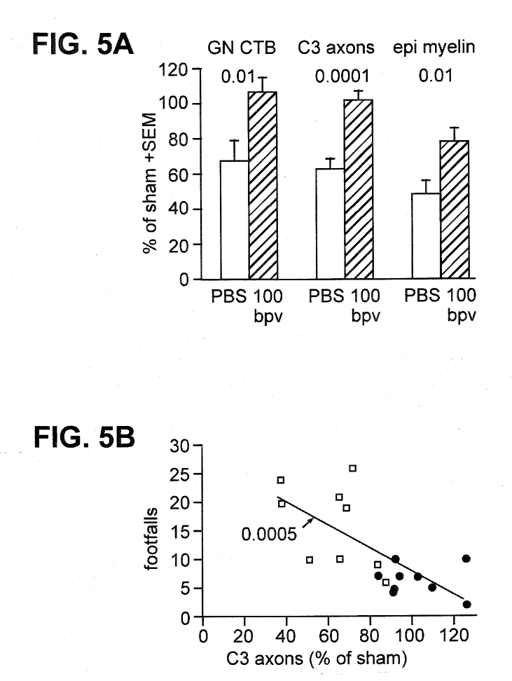

Four days after the last grid-walk test, the rats received injections of CTB tracer into the sciatic nerves (FIG. 1A). Three days later, i.e., 42 d after the injury and 14 d after the termination of the L5/6 intrathecal infusion, the bpV(phen)-treated rats had a more extensive CTB-labeled innervation of the gracile nucleus than the PBS-infused rats (FIG. 4C vs 4B), comparable with sham rats (FIG. 4A). With bpV(phen), the CTB value was 106+/−9% of sham compared with only 68+/−11% with PBS [p<0.01 vs bpV(phen); p<0.025 vs sham] (FIG. 5A). To confirm that the axons were rescued, the middle of the fasciculus gracilis, in which the hindlimb primary afferents originating from L3-L6 are located, was analyzed just caudal to the gracile nucleus at C3. Essentially all DRG neurons projecting to the gracile nucleus are the large ones that have myelinated axons, which were counted in semithin sections. The bpV(phen)-treated rats clearly had more myelinated sensory axons (FIG. 4F vs E) than the PBS-infused rats. Except for some white matter debris, the number of gracilis axons at C3 in the bpV(phen) group (FIG. 4F) appeared comparable with that of sham-operated rats (FIG. 4D). The number of myelinated axons was greater in the bpV(phen) group (102+/−5%) than in the PBS group (63+/−6%; p<0.0001) (FIG. 5A). The ratio of the CTB-labeled terminal fiber area and the C3 axons was the same in the PBS and bpV(phen) groups, evidence that bpV(phen) did not induce sprouting. This supports the interpretation that the bpV-(phen) infusion at L4 rescued the ascending sensory axons at the T9 injury site. The area of spared white matter in the dorsal column at the T9 injury epicenter was greater in the bpV(phen) group (79+/−7%; p<0.01) than in the PBS group (48+/−8%) (FIGS. 4I vs H, 5A), indicating that bpV(phen) can also permanently rescue myelin. When the treatment groups were combined, regression analyses of the hindlimb grid-walk performance against the histological parameters showed the clearest correlation with the number of axons in the fasciculus gracilis at C3 (FIG. 5B). The CTB-labeled gracile nucleus innervation and area of dorsal column white matter at the T9 injury epicenter also correlated inversely with the number of footfalls (p<0.05 and p<0.005, respectively). This further supports the interpretation that improved sensory axon and dorsal column white matter sparing contributed to improved sensorimotor function.

bpV(phen) Treatment Rescues Sensory-Evoked Potentials

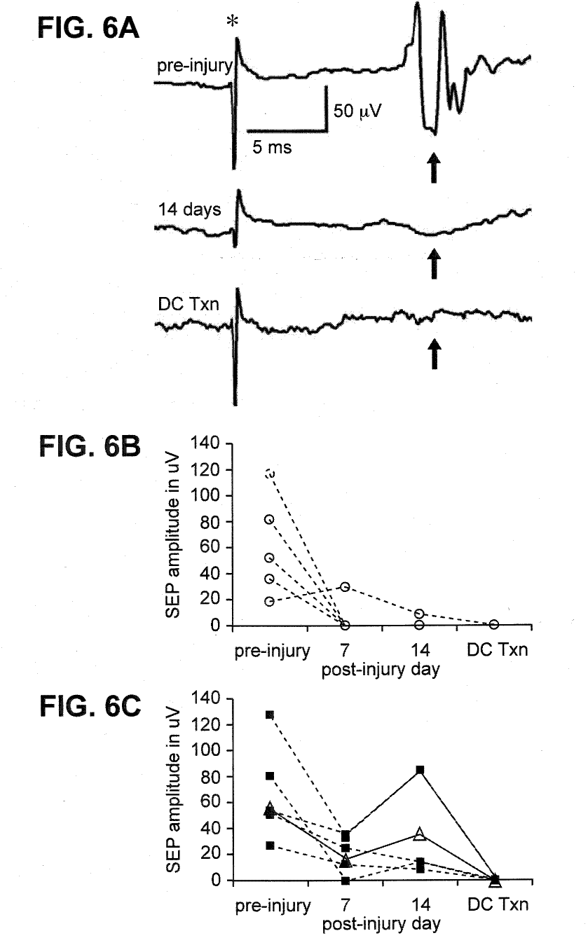

To directly test the function of the primary sensory afferents projecting to the gracile nucleus, SEPs were used, stimulating from the hindlimb and recording over the medulla (FIG. 1A). In preliminary experiments, SEPs did not disappear consistently after the 0.3 mm injury. Therefore, rats received a 0.4 mm T9 contusion and a 7 d lumbar infusion with PBS (n=5) or PBS containing 100 μM bpV(phen) (n=5) starting immediately after the injury. The recording electrodes had been placed and the existence of an SEP confirmed for each rat a few days before the contusion, with comparable average amplitudes of 61, 67, and 56 μV in the PBS, bpV(phen), and sham groups, respectively (FIG. 6). One week after the contusion, only one of five rats in the PBS group had an SEP (average, 6+/−6 μV) above the background noise, whereas four of five rats in the bpV(phen) group had an SEP (average, 21+/−7 μV), similar to the sham rat (16 μV). One additional week after termination of the infusion, only the same rat in the PBS group had a detectable SEP (group average, 2+/−2 μV), whereas all rats in the bpV(phen) group had an SEP (average, 30+/−18 vs 36 μV of the sham rat). To confirm that the rescued SEPs were mediated by the fasciculus gracilis, the latter was transected 1 mm caudal to the T9 contusion site. Two days later, none of the rats had SEPs. The lesion depth was confirmed in sagittal sections. Because of the dorsal column transection, these rats did not receive CTB tracing, and white matter sparing could not be assessed with confidence, also because of the sagittal plane of sectioning. Thus, L5/6 intrathecal bpV(phen) infusions rescue not only the structural integrity but also the function of the primary sensory dorsal column axons after a T9 contusion. The finding that the SEP was present after the 0.3 mm but not the 0.4 mm injury suggests that SEPs can be evoked with a minimum of somewhere between 15 and 31% of the dorsal column sensory axons (FIG. 1D).

Rescue of Axons, But not White Matter, Appears Mediated by Microvascular Protection

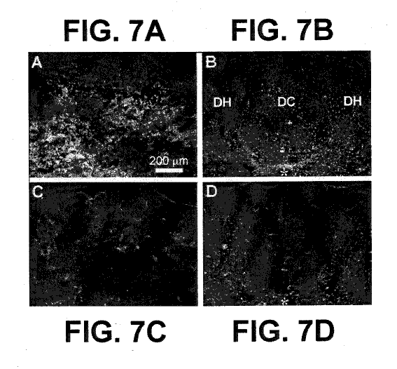

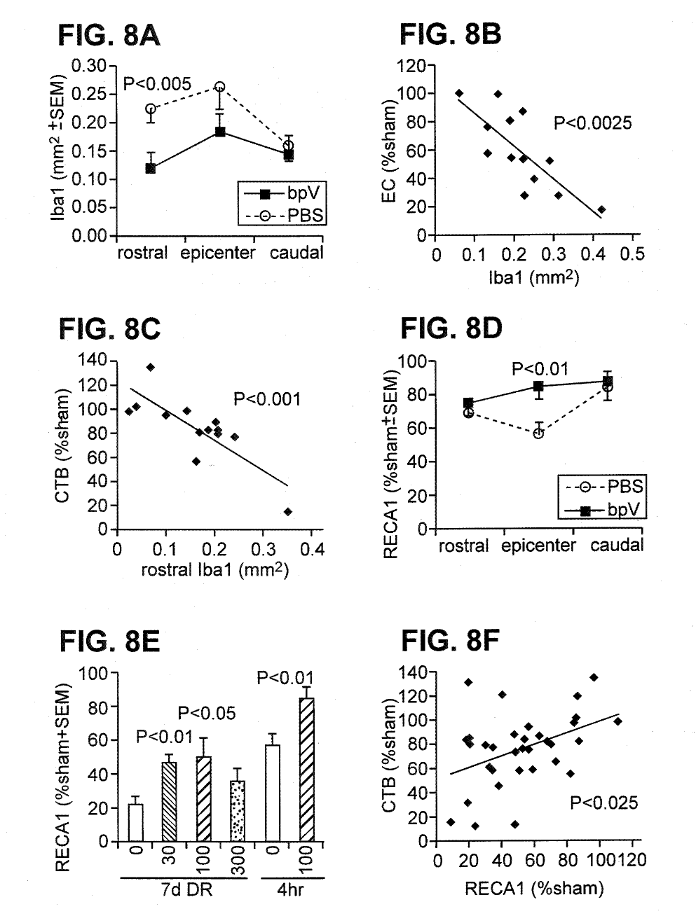

Inflammation is a major contributor to white matter loss after spinal cord injury. Therefore, to find a potential mechanism for the axon- and myelin-protective effects of bpV(phen), its effects on Iba1 immunostaining were measured as a marker for microglial/macrophage activation and inflammation. In the rats treated with bpV-(phen) for 7 d starting 4 h after the injury, sections through the injury epicenter clearly showed a reduction in Iba1 immunostaining (FIG. 7A vs B). Whereas, PBS-infused rats had clear infiltrates of activated macrophages, those infused with bpV(phen) had predominantly activated microglial cells. Moreover, whereas the PBS-treated rats showed degenerative changes in the gray and white matter architecture, the tissue in the bpV(phen)-treated rats appeared closer to normal. The area of Iba1-immunostained structures was significantly reduced at 1 mm rostral to the epicenter (p<0.005), whereas there was a trend of a reduction at the epicenter (p=0.053) but no difference at 1 mm caudal to the epicenter (p=0.49) (FIG. 8A). The extent of white matter loss in the dorsal column at the epicenter correlated to the extent of microglia/macrophage activation at the epicenter (FIG. 8B). The extent of inflammation 1 mm rostral to the injury correlated with the loss of CTB-labeled sensory projections in the gracile nucleus (FIG. 8C) but not with that seen at the epicenter (p=0.3) or caudal to the lesion (p=0.57). This suggests that the sensory axon sparing by bpV(phen) was not attributable to the reduced inflammation and that the reduction in inflammation rostral to the injury site might be an indirect result of reduced axon degeneration of the ascending projections. Conversely, the possibility that bpV(phen) affected inflammation at other times over the 7 d period cannot be excluded.

Another potential contributor to axon loss is ischemia caused by blood vessel loss. To test this possibility, the same sections were analyzed for the extent of endothelial cell/blood vessel sparing by immunostaining for RECA1. The spinal cord tissue architecture of PBS-treated rats was disturbed (FIG. 7C), whereas that of bpV(phen)-treated rats had an essentially normal appearance of both white and gray matter (FIG. 7D). Quantification of the blood vessels in the dorsal column at the epicenter showed clear loss in PBS-treated rats and sparing by bpV(phen) (FIG. 8D). The area was not significantly different between bpV(phen)-treated and sham-operated rats. Regression analyses revealed a trend between RECA1 and CTB (sensory axons; p=0.087) or dorsal column EC (myelin; p=0.085). Therefore, epicenter RECA1 was measured in additional 7 d postinjury groups, excluding those infused at the epicenter and those with an epicenter transection (SEP), which damaged the tissue. The bpV(phen) treatment (started immediately after the injury) increased the area of blood vessels in the dorsal column in those rats infused with 30 and 100 μM bpV(phen) (FIG. 8E). A regression analysis of all the 7 d infused rats [PBS and all doses of bpV(phen) combined] showed a correlation between RECA1-positive blood vessels in the dorsal column at the injury epicenter and CTB-labeled sensory projections (FIG. 8F). However, there was no correlation between RECA1- and EC-stained dorsal column epicenter white matter (p=0.98). This suggests that bpV(phen) rescued the sensory axons, but not their myelin, by rescuing dorsal column blood vessels in the injury epicenter.

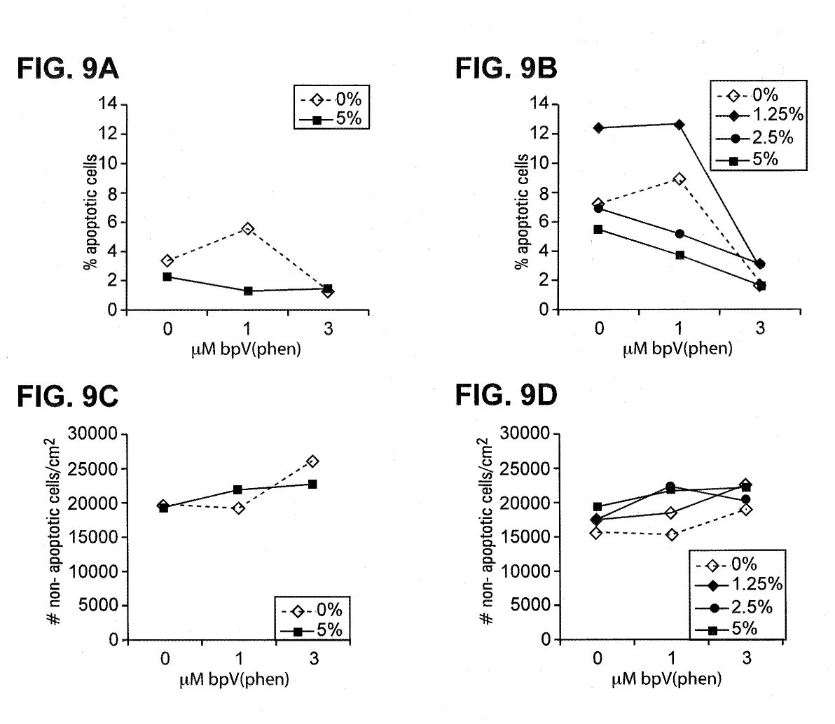

To determine whether bpV(phen) can directly promote survival of endothelial cells, it was tested on cultured human aortic endothelial cells deprived of serum. Six hours after plating, 2-3% of the cells showed the characteristic of apoptosis, nuclear fragmentation or condensation. With 3 μM bpV(phen), only ˜1.3% were apoptotic in both the 0 and 5% serum groups (FIG. 9A). Eighteen hours after plating under control conditions, between 5 and 13% of endothelial cells were apoptotic, whereas treatment with 3 μM bpV(phen) reduced apoptosis to ˜2-3% (FIG. 9B). The results from the 1 μM groups suggest that suboptimal doses of bpV(phen) can increase the survival-promoting effects of suboptimal concentrations of serum.

Discussion

The information provided herein provides a proof-of-principle that inhibition of PTPs can be used to reduce secondary degeneration and dysfunction of long-projecting axons, their myelin, and surrounding blood vessels, identifying a novel neurodegenerative mechanism after spinal cord injury. Moreover, clinically relevant treatment protocols can be applied with a small molecule to reduce the devastating outcomes of spinal cord injury.

The primary sensory system projecting from the sciatic nerve to the gracile nucleus was used because it is well defined and readily accessible to investigation. The hindlimb dorsal column-medial lemniscus system is important for tactile discriminatory aspects of sensation, including spatial and temporal characteristics. In humans and monkeys, lesions to this system causes disturbance of voluntary movement, including ataxia. Its degeneration, therefore, most likely contributes to functional sensory and motor deficits in humans with spinal cord injury. Here, the bpV(phen) treatment improved grid-walking performance, restoring this sensorimotor function to normal levels. The performance correlated well with the number of myelinated axons in the fasciculus gracilis, suggesting that sparing of these fibers contributed to the improved function. In rats, selective transection of the dorsal columns, excluding the dorsal corticospinal tract, cause deficits in a ladderwalking test. However, grid walking is also dependent on propriospinal function and on ventral pathways. The dorsal column at T9 also contains second-order propriospinal axons that do not project to the gracile nucleus, and the main propriospinal tract courses though the dorsolateral funiculus. Thus, it is possible that bpV(phen) also protected these and possibly other tracts. Electrophysiological data suggest that function of the primary sensory axons was protected by the bpV(phen) treatment, because the protection disappeared after selective transection of the fasciculus gracilis just caudal to the primary injury.

These results show that primary afferent dorsal column axons can be protected, e.g., fully protected, by bpV(phen) treatment after a moderate spinal cord injury of 0.3 mm displacement. The treatment also rescued the electrophysiological conduction across the injury site to the medulla after the more severe 0.4 mm injury. The protection by bpV(phen) seemed to add to some protection by the PBS infusion. This would be consistent with the findings that intrathecal PBS infusions increase NGF levels in the spinal cord and that NGF can reduce degeneration of stumps of directly transected dorsal column sensory axons. In contrast, the infusion at the level of the injury reduced the number of spared axons in a proportion of rats, possibly related to the presence of the catheter during and/or after the primary injury. The lumbar infusions reduce such a risk as well as the risk of peripheral side effects of any drug considered for spinal cord injury and can readily be applied to humans.

The bpV(phen) treatment also protected white matter in the dorsal columns and the total spinal cord at the injury site. The level of dorsal column white matter protection was not complete, suggesting that full functional recovery in rats can occur in the absence of completely intact myelination and/or that recovery is also affected by the plasticity of the spinal cord caudal to the injury. Inflammation is a major contributor to white matter loss after spinal cord injury. However, given the finding that bpV(phen) did not consistently reduce microglia/macrophage activation caudal and rostral to the injury epicenter, it is possible that the rescue of white matter by bpV(phen) indirectly reduced the inflammation. This would be consistent with the finding that the area of spared white matter inversely correlated with the Iba1 area at the epicenter. The finding that the CTB-traced projections inversely correlated with the inflammatory response only rostral to the injury site suggests that the reduced degeneration of the ascending axons in bpV(phen)-treated rats also indirectly reduced the inflammatory response. The possibility that bpV(phen) affected inflammation at other times over the 7 d period or infiltration of specific leukocytes and thereby reduced axon degeneration cannot be excluded.

Endothelial cells die over the first day after injury, and sensory axons seem to undergo secondary degeneration some time after the first day. Therefore, the axon-protective effects of bpV(phen) might indirectly be attributable to protection of endothelial cells and blood vessel integrity. In fact, the sections through the spinal cord injury epicenter showed that bpV(phen)-treated rats had an almost normal blood vessel network in sharp contrast to the PBS-treated rats. Moreover, the CTB-labeled sensory projections to the gracile nucleus correlated well with the extent of blood vessel sparing. Thus, protection of blood vessels during the first postinjury week might reduce ischemia, resulting in improved axonal survival. Of note is that the white matter sparing did not correlate with sparing of the vasculature, pointing to partially different mechanisms underlying white matter and axon degeneration. These observations identify PTPs as novel targets to rescue endothelial cells after neurotrauma.

The neuroprotective mechanisms of bpV(phen) most likely involve PTPs, because bpV(phen) is a well characterized and specific PTP inhibitor (Posner et al. (1994) J Biol Chem 269:4596-4604; Fantus et al. (1995) Mol Cell Biochem 153:103-112; and Drake et al. (1996) Endocrinology 137:4960-4968). bpV(phen) inhibits PTPs but not Ser/Thr phosphatases. Also, its insulin receptor kinase activation correlates with the tyrosine phosphorylation state of the receptor and its time dependent breakdown. bpV(phen) can inhibit the catalytic activity of purified yeast and mouse cdc25B PTP. Peroxovanadiums irreversibly inhibit PTPs by binding and oxidizing a critical cysteine in the catalytic domain (Posner et al. (1994) J Biol Chem 269:4596-4604; Bevan et al. (1995) Mol Cell Biochem 153:49-58; and Huyer et al. (1997) J Biol Chem 272:843-851). Moreover, bpV(phen)-induced protein tyrosine phosphorylation, and extracellular signal-regulated kinase (ERK) activation is reduced by antioxidants in cell lines. This raises the possibility that bpV(phen), like other metal-containing compounds, might oxidize proteins other than PTPs. However, bpV(phen) does not cause oxidative damage to DNA in C6 cells, does not change the glutathione concentration in rat cardiomyocytes, and does not cause death of H2O2-susceptible Madin-Darby canine kidney cells.

PTPs are a class of signaling proteins that can dephosphorylate tyrosine kinases (Johnson et al. (2003) Physiol Rev 83:1-24; Paul et al. (2003) Cell Mol Life Sci 60:2465-2482; and Alonso et al. (2004) Cell 117:699-711), suggesting that the protective mechanisms described herein might include enhancement of intracellular tyrosine kinases signaling. The kinases involved in the rescue effects after spinal cord injury might include neurotrophic receptors. bpV(phen) enhances ERK signaling in several systems. Peroxovanadiums seem to be broad-spectrum PTP inhibitors, enhancing tyrosine phosphorylation of multiple proteins in multiple systems (Sekar et al. (1996) Crit Rev Biochem Mol Biol 31:339-359; and Ruff et al. (1997) J Biol Chem 272:1263-1267). It is therefore surprising that bpV(phen) can have neuroprotective effects without disrupting cellular functions, even when given over extended treatment periods up to 4 weeks. Conversely, bpV(phen) appears to be somewhat selective. Its insulinomimetic actions in vivo are limited to fewer organs than those of the related bpV(pic) (Bevan et al. (1995) Mol Cell Biochem 153:49-58). bpV(phen) activates AP1 in macrophages, whereas bpV(pic) does not (Blanchette et al. (2007) J Leukoc Biol 81:835-844). Moreover, bpV(phen) has differential effects on intracellular signaling pathways, stimulating survival-promoting ERK but not c-Jun N-terminal protein kinase, STAT1a, or nuclear factor kB. Thus, it is possible that bpV-(phen) primarily, e.g., selectively, enhances function of proteins involved in axonal, oligodendrocyte, and endothelial survival and/or primarily reduces activity of PTPs involved in degeneration. Broad-spectrum inhibitors such as bpV(phen) might affect multiple neurotrophic and growth factor receptor signaling pathways, thereby potentially providing synergy.

Materials and Methods

Animals. Young adult female Sprague Dawley rats (n=111; 180-200 g; Harlan) were used. Experiments were approved by the University of Louisville Institutional Animal Care and Use Committee and conducted according to National Institutes of Health guidelines. All invasive procedures were performed under deep anesthesia with an intramuscular injection of 3.3 ml/kg mixture containing 25 mg/ml ketamine hydrochloride (Abbott Laboratories), 1.2 mg/ml acepromazine maleate (The Butler Company), and 0.25 mg/ml xylazine (The Butler Company) in 0.9% saline. Surgeries, behavioral measurements, sensory-evoked potentials (SEPs), and quantification of histological results were done by investigators blind to the treatment. Almost all experiments included at least one sham-operated rat (laminectomy at T9), and the ones with cholera toxin subunit B (CTB) labeling were combined for a total of n=7.

Spinal cord injury and intrathecal catheters. Polyethylene catheters were produced by stretching one end of a heated piece of polyethylene PE 60 tubing (Clay Adams, Becton Dickinson) to an outer diameter of ˜100 μm over a 1 cm length. A bead was made on the 4 mm nonstretched part, which was glued to the flow moderator of an Alzet osmotic pump (Durect). The assembly was gas sterilized by ethylene oxide gas. Alzet pumps (7 d; model 1007D) were filled under sterile conditions with sterile 0.1 M PBS or with PBS containing freshly dissolved bpV(phen) produced and nuclear magnetic resonance certified (Posner et al. (1994) J Biol Chem 269:4596-4604). The flow moderator was inserted for all but 2-3 mm into the pump, and the pumps were incubated for overnight at room temperature in sterile saline. For the 28 d infusions, every 7 d, new Alzet pumps were filled with fresh reagent and the previous subcutaneous pump was replaced.

Preparations for aseptic surgeries and postoperative care were performed as described previously (Baker et al. (2007) Exp Neurol 205:82-91). After a laminectomy at T9 vertebra level, the spinal column was stabilized in a frame with steel clamps inserted under the transverse processes. The contusion was applied with a set displacement of the spinal cord (0.2-0.6 mm depending on the experiment), using the Louisville Impactor System Apparatus (Louisville Impactor System Inc.). In short, the apparatus uses a laser distance sensor emitting a laser beam to measure the distance between the intact dura at T9 and the tip of the impactor head positioned 12.5 mm directly above it. The intended spinal cord tissue displacement is set by adjusting the vertical position of the stage on which the rat is secured, using a micrometer dial. The 2-mm-diameter impactor head is then accelerated by pneumatic cylinder at a velocity of 1.0 m/s over the first measured impactor-dura distance with the impactor-tissue contact duration preset at 200 ms. In one group, the catheters were introduced into the subarachnoid space through a small hole in the dura at T11, the tip was advanced to T9, and the infusion was started immediately after the contusion (Baker et al. (2007) Exp Neurol 205:82-91). All other infusions were made by inserting the catheter into the CSF at vertebral level L5/6, and the infusion was started immediately or 4 h after the contusion. That delayed time point was chosen because lumbar infusions could be started within that time in most humans with spinal cord injury in developed countries, increasing the clinical relevance of this study. The catheter was sutured to the muscles, and the pump was placed in a subcutaneous pocket. Gelfoam (Pharmacia & Upjohn) was used to seal the dura. Next, the rest of the flow moderator was inserted into the pump and the wound was closed in layers.

Function tests. Baseline values were determined a few days before the contusion. Sensorimotor function was tested by voluntary walking on a 45×45 inch grid with 1.5 inch holes. The hindlimb footfalls were called out by two investigators observing from different sides and recorded by a third. The test was completed after 90 s of walking was observed, and the total time was recorded. The potential development of pain was assessed by applying Semmes-Weinstein filaments to a shaved area of the trunk just rostral to the injury (at-level) and noting the filament size that induced a behavioral response (vocalization, orientation toward the stimulus with or without biting, avoidance by running away or moving out of the way, and freezing). For SEPs, the recording electrode made of silver was inserted into the midline epidural space just dorsal to the gracile nucleus, and an epidural reference electrode was placed over the olfactory bulb. These were anchored on the skull by dental cement. A ground electrode was placed subcutaneous on the back. Low electrical current (5 mA, 100 μs pulsed, 0.3 Hz) was applied to the calves through a ring stimulating electrode while the rats were awake and lightly restrained with a cone-shaped bag, and SEPs were recorded as described previously (Zhang et al. (2007) J Neurosci Methods 165:9-17). On each of the test days, 20 traces from each rat were averaged to determine the amplitude of the SEP for each rat. After the SEP session on day 14 after injury, the dorsal column was stereotactically transected by using the Vibraknife (Zhang et al. (2004) J Neurosurg 100:343-352), to a depth of just dorsal to the corticospinal tract. One or 2 d later, the disappearance of the SEPs was confirmed.

Anterograde tracing and histological procedures. In most experiments, hindlimb sensory projections were traced with CTB by bilateral injections into the sciatic nerves 3 d before analysis as described previously (Baker et al. (2007) Exp Neurol 205:82-91). Here, the hindlimbs were sprayed with Chew Guard (Veterinary Products Laboratories) to prevent autophagy. At the end of the experiment, the rats were processed for histological analyses as described previously (Baker et al. (2007) Exp Neurol 205:82-91). In short, after transcardiac perfusion with PBS and 4% paraformaldehyde, the spinal cord and attached brain were postfixed overnight and cryoprotected in 30% sucrose before 40 μm sagittal sections from the medulla and horizontally sections from the lumbar cord were collected in storage buffer in anatomical order. A 1 cm segment of injured thoracic spinal cord from each rat in an experiment was embedded in a block of freezing medium (Triangle Biomedical Sciences) and 20 20-μm transverse sections per 1 mm rostrocaudal distance were cut on a cryostat, thaw mounted onto charged microscope slides (catalog #12-550-15; Thermo Fisher Scientific), and stored at −20° C.

Anterogradely labeled sensory fibers and terminals were visualized in every third section through both gracile nuclei by DAB-based immunostaining for CTB as described previously (Baker et al. (2007) Exp Neurol 205:82-91). The lumbar sections were stained to ensure that the CTB injections were successful as evidenced by traced afferents and motor neurons. To detect white matter at the T9 epicenter, the transverse sections were stained with a modified eriochrome cyanine (EC) staining protocol (Rabchevsky et al. (2001) J Neurotrauma 18:513-522). The frozen slides were warmed up at 37° C. for 60 min, and freezing medium was removed. All other steps were at room temperature. The remaining freezing medium was removed by immersion in xylene, and the sections were hydrated through a graded alcohol range. Next, the slides were placed for 10 min into a solution consisting of 2 ml of 10% FeCl3 and 40 ml of 0.2% EC (Sigma) in 0.5% aqueous H2SO4, brought to a final volume of 50 ml with ddH2O. After rinsing in ddH2O and differentiation for 30 in 0.5% aqueous NH4OH, the reaction was terminated by rinsing in ddH2O. The sections were dehydrated and coverslipped with Entellan. The epicenter of maximal injury was determined by the rostrocaudal level with the least amount of spared white matter per transverse section. Adjacent sections were processed for double immunofluorescent staining to detect Iba1 (1:250; rabbit polyclonal antibody; catalog #019-19741; Wako Bioproducts) and RECA1 (1:40; mouse monoclonal antibody; catalog #MCA970; Serotec), using Alexa594-conjugated donkey anti-rabbit and Alexa488-conjugated donkey anti-mouse IgG as secondary antibodies (1:500; Invitrogen). To detect individual myelinated axons of the fasciculus gracilis, a section of the C3 spinal cord was stained with toluidine blue and prepared for semithin sectioning by embedding in Epon and cutting 2-μm-thick transverse sections. These were coverslipped in DPX.

Endothelial cell cultures. Human aortic endothelial cells (Lonza Walkersville) were plated at 5×10⁴ cells per well in 24 well polystyrene plates and incubated with different concentrations of bpV(phen) for 6 or 18 h at 37° C. in DMEM/Ham's F-12 (BioWhittaker) plus EGM-2 growth factor supplements [EGM-2 SingleQuots (Lonza Walkersville) containing hydrocortisone, human epidermal growth factor, FBS, vascular endothelial growth factor, human basic fibroblast growth factor, R3-IGF-1, ascorbic acid, heparin, and gentamicin/amphotericin-B] and 10-20% fetal calf serum. To induce apoptosis, these cultures were serum deprived by adding 0, 1.25, 2.5, or 5% fetal calf serum, instead of the normal 10-20%. Afterward, the endothelial cells were fixed in 4% paraformaldehyde for 2 h, the nuclei were stained with 2.5 μg/ml DNA dye Hoechst 33258 (bis-benzimide; Sigma) in PBS containing 0.1% Triton X-100 for 30 min, and the cells were covered with Fluoromount-G (Southern Biotechnology) to reduce fluorescence-induced fading (Invitrogen).

Quantitative measurements and statistics. The extent of sensory innervation of the gracile nucleus was determined by measuring the CTB-labeled terminal fiber area in every third section as described previously (Baker et al. (2007) Exp Neurol 205:82-91). In short, images of every third section through the right and left gracile nuclei were analyzed using the density slice feature of the Scion Image software. The areas for all sections through both gracile nuclei were summed for each rat. Images of EC-stained sections were captured, and the positive areas were measured with Scion Image. The injury epicenter was defined by the greatest loss of EC staining in the transverse sections. Measurements of the area of EC, Iba1, or RECA1 staining were made in three sections (spaced 100 μm) per 1 mm rostrocaudal distance along the 1 cm spinal cord section and averaged for each distance. Measurements within the dorsal column were possible for EC and RECA1 staining. Iba1 measurements were made in the entire transverse plane of the spinal cord, because it was difficult to determine the borders between the dorsal column and dorsal horns. The number of spared myelinated axons at C3 was counted in semithin sections using a 63× oil objective in 15.7×15.7 μm grids placed over the middle part of each fasciculus gracilis (see FIG. 4D-4F). The amplitudes of the SEPs were measured at the expected position seen between 11 and 15 ms after the stimulus artifact. Waves that could not be distinguished from the noise were assigned a zero amplitude. To determine the effects of bpV(phen) on endothelial apoptosis, the percentage of cultured endothelial cells with condensed or fragmented Hoechst-stained nuclei was determined by counting 10 fields per well using a 40× objective and yielding between 350 and 500 total nuclei.

Statistical analysis was performed with StatView (SAS Institute) and Microsoft Excel. Data was compared between groups using either ANOVA in cases of multiple groups or repeated measures or by the t test. Statistical significance was determined by a p value of <0.05. A trend was indicated by a p value of >0.05 and <0.1.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

What is claimed is:

1. A therapeutic method for treating traumatic neural injury in a mammal, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

2. A therapeutic method for treating a degenerative disorder in a mammal, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

3. A therapeutic method for pre-treating a mammal prior to surgery to prevent injury to nerves, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

4. A therapeutic method for preventing damage to endothelial cells, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

5. A therapeutic method for preventing damage to blood vessels, comprising administering to a mammal in need of such therapy an effective amount of a neuroprotective compound, wherein the neuroprotective compound is a peroxovanadium compound, a peroxovandium derivative, or a compound with inhibitory activity of protein tyrosine phosphatases.

6. The method of claim 1, wherein the neuroprotective compound is potassium bisperoxo(1,10-phenanthroline)oxovanadate (V) [bpV(phen)].

7. The method of claim 1, wherein the neuroprotective compound is potassium bisperoxo(pyridine-2-carboxylato)oxovanadate(V) [bpV(pic)], pervanadate, periodinate or dephostatin.

8. The method of claim 1, wherein the neuroprotective compound is administered directly into or around the injured tissue, is administered through delivery into the cerebrospinal fluid, is administered onto or directly into the eye or is administered intravenously.

9. The method of claim 1, wherein the neuroprotective compound is administered at a concentration of about 100 μM.

10. The method of claim 1, wherein the neuroprotective compound is administered at a concentration of less than 100 mM.

11. The method of claim 1, wherein the neuroprotective compound is administered at a concentration of about 30 to 300 μM.

12. The method of claim 1, wherein the neuroprotective compound is administered at a concentration of less than about 100 μM.

13. The method of claim 1, wherein the neuroprotective compound is administered within about 0-48 hours of injury.

14. The method of claim 13, wherein the neuroprotective compound is administered within about 2-24 hours of injury.

15. The method of claim 14, wherein the neuroprotective compound is administered within about 3-12 hours of injury.

16. The method of claim 15, wherein the neuroprotective compound is administered within about 3-5 hours of injury.

17. The method of claim 1, wherein the mammal is a human, cat, dog, horse, donkey, mule, cow, sheep, goat, or camel.

18. The method of claim 1, wherein the neuroprotective compound is administered for a period of about one to four weeks.

19. The method of claim 1, wherein the traumatic neural injury is a spinal cord injury, brain injury, a peripheral nerve injury, an eye injury affecting the optic nerve fibers, or a skin burn.

20. The method of claim 19, wherein the traumatic neural injury is a traumatic spinal cord injury.

21. The method of claim 1, wherein the traumatic neural injury is caused by an ischemic or hemorrhagic stroke.

22. The method of claim 2, wherein the degenerative disorder is a neuron, axon, or myelin disorder.

23. The method of claim 2, wherein the degenerative disorder is an oligodendrocyte disorder.

24. The method of claim 2, wherein the degenerative disorder is multiple sclerosis or peripheral neuropathy.
